(19) United States
(12) Patent Application Publication
(10) Pub. No.: US 2024/0115758 A1
(43) Pub. Date: Apr. 11, 2024

(54) FLOWABLE HYDROGEL HYDROCOLLOID COMPOSITE SEALANT

(71) Applicant: Ethicon, Inc., Raritan, NJ (US)

(72) Inventors: Salim Ghodbane, Piscataway, NJ (US); Sridevi N. Dhanaraj, Raritan, NJ (US); Ashley DeAnglis, Somerville, NJ (US); Gary Zhang, Raritan, NJ (US)

(21) Appl. No.: 17/937,642

(22) Filed: Oct. 3, 2022

(51) Int. Cl. A61L 24/00; A61L 24/04; A61L 24/10

(52) U.S. Cl. CPC A61L 24/0031; A61L 24/043; A61L 24/104; A61L 2430/34

(57) ABSTRACT

Provided herein are hydrogel-hydrocolloid sealant compositions for use as soft-tissue sealants. The compositions can include a cross-linkable electrophilic component; a cross-linkable nucleophilic component; a swellable filler material including gelatin; and, a buffer having a pH in the range of about 9.0 to about 10.0. The compositions can have a first fluid state and a second crosslinked state, and in the crosslinked state, the electrophilic and nucleophilic components can crosslink to form a crosslinked hydrogel network where the swellable filler material including gelatin is disposed within the crosslinked network. Also provided are systems and methods of manufacturing the sealant compositions as well as methods of repairing soft tissue defects including applying the sealant compositions disclosed herein.

### FIELD OF DISCLOSURE

The present disclosure is directed to crosslinking hydrogel-hydrocolloid sealant compositions, as well as systems and methods of manufacturing for same. The present disclosure additionally describes methods of treatment using these compositions.

### BACKGROUND

Image-guided percutaneous transthoracic needle biopsy (PTNB) is an established procedure for patients with suspected pathologic processes, such as bronchogenic carcinoma. The goal of the procedure is to obtain tissue for cytologic or histologic examination. The procedure is typically performed with image guidance by a radiologist. Imaging modalities utilized include fluoroscopy, computed tomography (CT), and ultrasound. Ultrasound is the safest, quickest, and least expensive method; however, it is only useful with very superficial samples. When lesions are not suitable for ultrasound, CT is the preferred imaging modality.

PTNB is classified according to the type of needle. Fine needle aspiration biopsy is performed to provide cytological specimens and larger diameter cutting needles to produce histological specimens. Historically, cutting needles have been associated with a relatively high incidence of complications but, with the introduction of automated cutting needles, recent studies have demonstrated comparable complication rates between fine needle aspiration and cutting needles.

During PTNB, an aspiration (18-22 gauge) or cutting needle (14-20 gauge) is placed under image guidance for sample recovery. A coaxial technique may be used to allow for multiple passes within the lung tract and to reduce the number of pleural punctures. In this technique, a thin-walled introducer needle (13-19 gauge) is first inserted, localized to the lesion, and subsequently the aspiration or cutting needle is inserted.

Although the procedure is considered safe and effective, the incidence of pneumothorax is still significant with ranges from 12 to 61%, with 2 to 15% requiring a chest drain. The risk of pneumothorax increases significantly if the lesion is not adjacent to the pleura. Most complications occur immediately or within the first hour following the biopsy. Therefore, following the procedure, the patient is placed in a puncture-site-down position and remain under supervision for at least 1 hour. The patient may present shortness of breath, chest pain, and hypoxia. Most acutely symptomatic pneumothoraxes are detectable via chest radiograph. Patients observed with pneumothoraxes are administered oxygen to speed resorption of pneumothoraxes.

Transbronchial needle aspiration (TBNA) is a minimally invasive technique allowing for the sampling of mediastinal nodes. When integrated with endobronchial ultrasonography (EBUS), accurate definition of mediastinal structures is possible. Modern devices integrate an ultrasonic bronchoscope into the needle allowing for real time visualization of the area of interest. The diagnostic yield of EBUS-TBNA in lung cancer screening has been reported with a sensitivity as high as 95.7%. As a result, EBUS-TBNA is becoming widely adopted as the standard of care for sampling mediastinal lymph nodes.

EBUS devices consists of a transducer and a processor. The transducer produces and receives sound waves. The processor integrates the reflected sound, generating images. The probe includes a balloon which can be inflated to improve contact with the airways. EBUS-TBNA devices include an ultrasound linear processing array and a retractable needle. EBUS-TBNA was originally performed with a dedicated 22-gauge aspiration needle; however, larger 21-gauge needles were introduced more recently. EBUS-TBNA are carried out in the proximal lumen of level 9 bronchi, as they are restricted by the outer diameter of the bronchoscope (6.9 mm). Although complications are very low in EBUS-TBNA, incidence of pneumothorax is still significant. The rate of pneumothorax has been estimated from 0.53% to 16.7% following EBUS-TBNA.

In most institutions worldwide, the choice between TBNA or PTNB still lacks a standardized strategy. The choice is typically influenced by environmental factors such as operator experience or institution resources. There is not an established algorithm based on clinical scenarios. However, PTNB is typically preferred for lesions near the visceral pleura and TBNA is preferred for those near the airways.

Patients in whom enlarging pneumothoraxes are observed must be treated with the placement of a chest tube. However, there is no universally accepted approach to reduce pneumothorax rate. Multiple solutions have been employed to reduce the incidence of pneumothoraxes. Several authors have investigated techniques, including the rapid roll over and deep expiration and breath-hold technique, to reduce the rate of pneumothorax but these techniques have only shown mild/moderate effects, with a risk reduction of 0.1-15.7%.

Therefore, others have investigated the instillation of various sealant materials into the track, including autologous blood clot, fibrin glue, and gelatinous foam, but none have achieved widespread use in daily practice. These methods have also suffered from variable results, possibly a result of operator-dependence and variations in practice. Autologous blood clot has demonstrated moderate efficacy but suffers from the long preparation times in the operating room. Although fibrin glue and gelatin techniques have demonstrated some promising published data, they have not been studied extensively.

Liquid synthetic sealants such as those derived from reactive polyethylene glycols (example: PEG-Amine and PEG-succinimidyl glutarate (SG)) or biological sealants (example: fibrin sealant) can only be effective in sealing lung tissue tracts if they could crosslink sufficiently without being disturbed. While this would theoretically lead to significant adhesive strength (via covalent crosslinking and/or mechanical interlocking, and cohesive strength), the positive pressure of the lung encountered during surgery, in combination with the low density and viscosity of the liquid sealants results in the inability of the sealant to crosslink completely. The sealant would be disturbed via expulsion of air from the tract and/or unintentional foaming which creates a path of least resistance to form, resulting in its inability to seal the air leak.

More recently, a synthetic polyethylene glycol plug has been developed as part of the BioSentry Tract Sealant System (Angiodynamics). In a randomized, multicenter clinical trial, BioSentry resulted in the absence of pneumothorax in 85% of patients which was statistically greater than the control group (69%). However, the solid nature of the plug induces only a foreign body giant cell reaction and an encapsulation of the hydrogel by 21 days (see images below). A more porous plug would lead to a reduced foreign body reaction and more rapid healing.

WO 2008/016983 relates to wound sealing compositions comprising first and second cross-linkable components and at least one hydrogel-forming component. The compositions may also include rapidly acting materials, for example a tissue sealant, and the compositions exhibit minimal swelling properties. The first and second cross linkable components may each, for example, be polyethylene glycols, and the hydrogel forming component may, for example, be gelatin that may comprise subunits having sizes ranging from about 0.01 mm to about 5 mm when fully hydrated and have an equilibrium swell ranging from about 400% to about 5000%. The first and second components react under in-vivo conditions to form a cross-linked matrix, while the hydrogel-forming component rapidly absorbs the biological fluid coming through the tissue breach, as well as strengthens the resultant physical sealant matrix barrier formed as the first and second components cross-link.

Thus, there is a need in the art for improved sealant compositions that can effectively seal a pleural tract.

### SUMMARY

The present disclosure describes a hydrogel-hydrocolloid expandable soft tissue sealant composition including a cross-linkable electrophilic component; a cross-linkable nucleophilic component; a swellable filler material including gelatin; and, a buffer having a pH in the range of about 9.0 to about 10.0. The sealant includes a first fluid state and a second crosslinked state, and, in the crosslinked state, the electrophilic and nucleophilic components crosslink to form a crosslinked hydrogel network, and the swellable filler material including gelatin is disposed within the crosslinked network.

According to certain embodiments, the gelatin is thermally crosslinked, a prewet gelatin fluid, or dry gelatin particles. In additional embodiments, the swellable filler material includes a blend of prewet gelatin fluid and dry gelatin particles, and in further embodiments the swellable filer material is present in the in the composition in an amount in the range of 50 mg/ml to about 10 mg/ml.

In still further embodiments, in the crosslinked state the composition has a terminal bubble velocity, and the terminal bubble velocity of the crosslinked composition is at least 75% less than a terminal bubble velocity of a composition including the electrophilic and nucleophilic components in the absence of the swellable filler. In additional embodiments, the terminal bubble velocity of crosslinked the composition is at least 90% less than a terminal bubble velocity of a composition including the electrophilic and nucleophilic components in the absence of the swellable filler.

According to certain embodiments, the electrophilic component includes a multi-arm polyethylene glycol (PEG) based polymer, and the nucleophilic component includes a multi-arm PEG polymer containing at least one reactive amine group. In further embodiments, the electrophilic component includes a PEG N-hydroxysuccinimide activated ester (PEG-NHS), for example, PEG-succinimidyl glutarate ester (PEG-SG). In alternative embodiments, at least one of the cross-linkable electrophilic component and the cross-linkable nucleophilic component is a biological compound, including embodiments, where both the cross-linkable electrophilic component and the cross-linkable nucleophilic component are biological compounds, for example, where the electrophilic compound is thrombin and the nucleophilic compound is fibrinogen.

In still further embodiments, the sealant composition can have a crosslinked state where the composition is solid, and further where the composition is lyophilized. In additional embodiments of the sealant composition, the swellable filler material is configured to expand in volume in the crosslinked state such that the composition has a crosslinked expandable state, and in the crosslinked expandable state, the composition is configured to provide a fluid tight seal at a pressure differential of up to 25 cm of water.

The present disclosure further describes a system for forming a hydrogel-hydrocolloid expandable soft tissue sealant including: a first container containing a cross-linkable electrophilic component; and, a second container containing a cross-linkable nucleophilic component; and, a swellable filler material including gelatin, where the swellable filler material is disposed in either the first container, the second container, or both, where the first container and the second container are configured to connect in fluid communication with one another. The cross-linkable electrophilic component and the cross-linkable nucleophilic component are configured to form a cross-linked hydrogel network upon admixture between the first container and the second container, and the swellable material is disposed within the crosslinked hydrogel network.

In certain embodiments, the swellable material is disposed in the first container, the swellable material is disposed in the second container, or the swellable material is disposed in each of the first and second container. In certain embodiments, the swellable material is a prewet gelatin fluid, the swellable material is dry gelatin particles, or the swellable material includes both prewet gelatin fluid and dry gelatin particles. In additional embodiments, both the first and second containers contain prewet gelatin fluid; in further alternative embodiments, the first container contains prewet gelatin fluid and the second container contains dry gelatin particles; and, in still further alternative embodiments, the first container contains dry gelatin particles, and the second container contains prewet gelatin fluid.

According to the present disclosure, a method of repairing a soft tissue defect is described including:
applying a hydrogel-hydrocolloid expandable sealant composition to a soft tissue defect, the composition containing a first cross-linking component and a second crosslinking component, and a swellable filler material including gelatin;
where the soft tissue defect extends from an outer surface of the soft tissue to an inner surface of the soft tissue, and where the inner surface defines a void containing a fluid exerting a positive pressure in the direction from the void to the outer surface; and,
exposing the sealant composition to an aqueous fluid such that the composition will crosslink and swell such that the composition will fluidly seal the soft tissue defect with adhesion to the soft tissue surface and expansive forces across the defect.

### BRIEF DESCRIPTION OF THE DRAWINGS

FIGS. 1A, 1B and 1C are graphical representations of ultimate extension, ultimate stress, and elastic modulus measurements, respectively, of formulations according to the present disclosure;

FIG. 2 is a graphical representation of terminal bubble rise velocity values of several samples of varying concentrations and types of gelatin mixed in a non-functional PEG solution; and,

FIGS. 3A, 3B, 3C and 3D are flowcharts depicting several systems and methods of manufacturing sealant compositions according to the present disclosure; and

FIG. 4 is a comparison graph showing swelling properties of two materials.

### DETAILED DESCRIPTION

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Further, reference to values stated in ranges includes each and every value within that range. It is also to be appreciated that certain features of the invention, which, for clarity, are described herein in the context of separate embodiments, may also be provided in combination in a single embodiment. Conversely, various features of the invention that are, for brevity, described in the context of a single embodiment, may also be provided separately or in any subcombination.

As used herein, “biocompatible” means compatible with living tissue or a living system by not being toxic, injurious, or physiologically reactive therewith and not causing immunological rejection thereby.

As used herein, “biologically absorbable” or “resorbable” means capable of degradation in the body to smaller molecules having a size that allows them to be transported into the blood stream. Such degradation and transportation gradually remove the material referred to from the site of application. For example, gelatin can be degraded by proteolytic tissue enzymes to absorbable smaller molecules, whereby the gelatin, when applied to tissue, typically is absorbed within about 4-6 weeks, and when applied to bleeding surfaces or mucous membranes, typically liquefies within 2 to 5 days.

As used herein, “hemostasis” means the process by which bleeding diminishes or stops. During hemostasis three steps occur in a rapid sequence. Vascular spasm is the first response as the blood vessels constrict to allow less blood to be lost. In the second step, platelet plug formation, platelets stick together to form a temporary seal to cover the break in the vessel wall. The third and last step is called coagulation or blood clotting. Coagulation reinforces the platelet plug with fibrin threads that act as a “molecular glue.” Accordingly, a hemostatic material or compound is capable of stimulating hemostasis.

As used herein, “pneumostasis” means to deliver a material(s) to pulmonary tissue to close or seal one or more air leaks.

As used herein, “prewet gelatin” refers to gelatin particles in aqueous solutions (e.g., SURGIFLO) that meet the specifications defined by the United States Pharmacopeia (e.g., USP 29).

As used herein, “dry gelatin” refers to dry gelatin particle powder (e.g., SURGIFOAM powder) that meet the specifications defined by the United States Pharmacopeia (e.g., USP 29).

As used herein the phrase “consisting essentially of” is intended to define the scope of a claim as including the recited components, compounds, substances, materials, or steps, and additionally include any components, compounds, substances, materials, or steps that do not materially affect the basic characteristics of the claimed invention.

The present disclosure describes a hydrogel-hydrocolloid expandable soft tissue sealant composition including a cross-linkable electrophilic component; a cross-linkable nucleophilic component; a swellable filler material including gelatin; and, a buffer having a pH in the range of about 9.0 to about 10.0. The sealant includes a first fluid state and a second crosslinked state, and, in the crosslinked state, the electrophilic and nucleophilic components crosslink to form a crosslinked hydrogel network, and the swellable filler material including gelatin is disposed within the crosslinked network.

Gelatin

Both the prewet gelatin particles and the dry gelatin particles are made from absorbable gelatin sponges that meet the specifications defined by the United States Pharmacopeia (e.g., USP 29). The porous structure and the degree of cross-linking of the sponges are measured by water absorption and by digestibility following the USP methods. For each type of particles, the sponge should absorb not less than 35 times its weight of water, and the average digestion time by pepsin is not more than 75 minutes.

The prewet gelatin particles are made by mechanically milling the sponges into fine particles, and mixing the particles with an aqueous solution, for example saline solution. They may have a particle size D90 of less than 1000 microns. That is, 90% of the prewet gelatin particles may have a diameter less than 1000 microns. The degree of cross-linking in the prewet gelatin particles is such that they have a digestion time of more than 30 minutes, but not more than 75 minutes measured by the USP digestibility test (for e.g., as referenced in USP 34 monograph). The prewet gelatin particles have a higher degree of cross-linking degree than dry gelatin particles

The dry gelatin particles are made by milling the gelatin sponges. They may have a particles size D90 of less than 2000 microns. That is, 90% of the dry gelatin particles may have a diameter less than 2000 microns. The degree of cross-linking in the dry gelatin particles is such that they have a digestion time of less than 30 minutes measured by the USP digestibility test. The dry gelatin particles when mixed with aqueous solution have a higher degree of swelling (absorbing liquid at least 35 times its own dry weight as described in USP 34 monograph) than the prewet gelatin particles.

According to certain embodiments, the gelatin is thermally crosslinked. In still further embodiments, the gelatin is a prewet gelatin fluid, or dry gelatin particles. In additional embodiments, the swellable filler material includes a blend of prewet gelatin fluid and dry gelatin particles, and in further embodiments the swellable filer material is present in the in the composition in an amount in the range of 50 mg/ml to about 10 mg/ml.

In still further embodiments, in the crosslinked state the composition has a terminal bubble velocity (as defined below), and the terminal bubble velocity of the crosslinked composition is at least 75% less than a terminal bubble velocity of a composition including the electrophilic and nucleophilic components in the absence of the swellable filler. In additional embodiments, the terminal bubble velocity of crosslinked the composition is at least 90% less than a terminal bubble velocity of a composition including the electrophilic and nucleophilic components in the absence of the swellable filler.

According to certain embodiments, the electrophilic component includes a multi-arm polyethylene glycol (PEG) based polymer, and the nucleophilic component includes a multi-arm PEG polymer containing at least one reactive amine group.

The electrophilic and nucleophilic reactive compounds that form the cross-linked structure of the hydrogel sealant are known in the art, and can include both synthetic polymers, such as multi-arm polyethylene glycol (PEG) based polymers, and natural substances, as well as combinations thereof. Suitable multi-arm PEGs can include 2, 3, 4, 6 or 8 multi-arm PEGs.

According to certain embodiments, synthetic polymer can include polymers having activated esters. such as from the class of compounds of PEG-N-hydroxysuccinimide (PEG-NHS), PEG-aldehydes, PEG-acrylates, Carboxyl-PEGs, and 4-arm vinyl-PEGs. According to further embodiments, a non-exhaustive list of suitable electrophilic compounds can include 4-arm-PEG-succinimidyl glutarate (SG), 4-arm-PEG-succinimidyl valerate, 4-arm-PEG-succinimidyl carbonate, 4-arm-PEG-succinimidyl succinate, 4-arm-PEG-succinimidyl butanoate, 4-arm-PEG-succinimidyl succinamide, 4-arm-PEG-succinimidyl propionate, 4-arm-PEG-sulfosuccinimidylglutarate (SG), 4-arm-PEG-sulfosuccinimidylvalerate, 4-arm-PEG-sulfosuccinimidylcarbonate, 4-arm-PEG-sulfosuccinimidylsuccinate, 4-arm-PEG-sulfosuccinimidylbutanoate, 4-arm-PEG-sulfosuccinimidylsuccinamide, 4-arm-PEG-sulfosuccinimidylpropionate, and 4-arm-PEG-isocyanate, 4-arm-PEG-imidoester, 4-arm-PEG-maleimide, 4-arm-PEG-acetic acid, 4-arm-PEG-propanoic acid, 4-arm-PEG-butanoic acid, 4-arm-PEG-hexanoic acid, and 4-arm-PEG-vinylsulfone. Other examples include 2 arm, 3 arm, 6 arm, and 8 arm-PEGs of the exemplary 4-arm compounds listed above.

As previously described, the electrophilic compound can also include a blend of natural and synthetic components.

In certain embodiments, the nucleophilic compounds include natural compounds, such as albumin, or fibrinogen. In certain additional embodiments, the nucleophilic compounds can include a synthetic polymer, preferably a multi-arm polymer. In further embodiments, the nucleophilic compound contains at least one reactive amine group, such as, for example, a 4-arm PEG-amine or PEG-hydrazide, or 4-arm PEG-thiol.

In preferred embodiments, the electrophilic component includes a PEG N-hydroxysuccinimide activated ester (PEG-NHS), for example, PEG-succinimidyl glutarate ester (PEG-SG). In alternative preferred embodiments, at least one of the cross-linkable electrophilic component and the cross-linkable nucleophilic component is a biological compound, including embodiments, where both the cross-linkable electrophilic component and the cross-linkable nucleophilic component are biological compounds, for example, where the electrophilic compound is thrombin and the nucleophilic compound is fibrinogen.

In still further embodiments, the sealant composition can have a crosslinked state where the composition is solid, and further where the composition is lyophilized. In additional embodiments of the sealant composition, the swellable filler material is configured to expand in volume in the crosslinked state such that the composition has a crosslinked expandable state, and in the crosslinked expandable state, the composition is configured to provide a fluid tight seal at a pressure differential of up to 30 cm of water.

The present disclosure further describes a system for forming a hydrogel-hydrocolloid expandable soft tissue sealant including: a first container containing a cross-linkable electrophilic component; and, a second container containing a cross-linkable nucleophilic component; and, a swellable filler material including gelatin, where the swellable filler material is disposed in either the first container, the second container, or both, where the first container and the second container are configured to connect in fluid communication with one another. The cross-linkable electrophilic component and the cross-linkable nucleophilic component are configured to form a cross-linked hydrogel network upon admixture between the first container and the second container, and the swellable material is disposed within the crosslinked hydrogel network.

In certain embodiments, the swellable material is disposed in the first container, the swellable material is disposed in the second container, or the swellable material is disposed in each of the first and second container. In certain embodiments, the swellable material is a prewet gelatin fluid, the swellable material is dry gelatin particles, or the swellable material includes both prewet gelatin fluid and dry gelatin particles. In additional embodiments, both the first and second containers contain prewet gelatin fluid; in further alternative embodiments, the first container contains prewet gelatin fluid and the second container contains dry gelatin particles; and, in still further alternative embodiments, the first container contains dry gelatin particles, and the second container contains prewet gelatin fluid.

According to the present disclosure, a method of repairing a soft tissue defect is described including:
applying a hydrogel-hydrocolloid expandable sealant composition to a soft tissue defect, the composition containing a first cross-linking component and a second crosslinking component, and a swellable filler material including gelatin;
where the soft tissue defect extends from an outer surface of the soft tissue to an inner surface of the soft tissue, and where the inner surface defines a void containing a fluid exerting a positive pressure in the direction from the void to the outer surface; and,
exposing the sealant composition to an aqueous fluid such that the composition will crosslink and swell such that the composition will fluidly seal the soft tissue defect with adhesion to the soft tissue surface and expansive forces across the defect.

### EXAMPLES

### Example 1—Effect of pH on Sealant Formulation Preparation

PEG Amine/Surgifoam Preparation

5 mL, 114 mg/mL PEG-Amine4-5k was added to one unit of Surgifoam® powder and uniformly distributed by shaking the powder until a moist ball formed. This was tested at two pH ranges of the PEG-Amine formulation: [9.0-9.5], and [10.5-11]. When mixed with the PEG-Amine solution, the Surgifoam® powder formed a more liquid and soft paste at pH=9.0 than at pH≥9.5. The powder was more easily workable at pH=9.0. The samples were transferred to a 20 mL luer lock syringe.

PEG SG/Surgiflo Preparation

750 mg PEG-SG4-10k was dissolved in 5 mL, 100 mM carbonate buffer (pH=9.0) for 5 minutes. The PEG-SG4-10k was loaded into a 20 mL luer lock syringe and homogenously distributed into one Surgiflo® unit using the dual syringe exchange method.

pH: 10.5/11.0

Once the Surgifoam®/PEG-Amine at pH=10.5 and 11.0 and Surgiflo®/PEG-SG components were thoroughly mixed using the dual syringe method with 20 passes, the sealant crosslinked within the syringes before 2 passes could be completed and were therefore considered to crosslink too quickly.

pH: 9.0/9.5

Once the Surgifoam®/PEG-Amine at pH=9.0 and 9.5 and Surgiflo®/PEG-SG components were thoroughly mixed using the dual syringe method with 20 passes the sealant could be expressed after 3 and 2 minutes at pHs 9.0 and 9.5, respectively. Once fully crosslinked, the sealant was stiffer at pH=9.0 than pH=9.5.

### Example 2—Comparison of Tensile Properties of Surgiflo with Thrombin and Surgiflo with Reactive PEG Matrix

Formulations:

A. Surgiflo with Thrombin

Surgiflo with Thrombin was prepared according to the FDA approved instructions for use (IFUs) for the Surgiflo Hemostatic Matrix Kit. Sterile water for injection syringe was connected to the thrombin vial and the entire amount of water for injection was added to the thrombin vial and mixed with the thrombin until a clear solution formed. The connected syringe was used to draw the entire contents of the solution out of the thrombin vial and into the syringe. The contents of the syringe were deposited into a sterile transfer cup for further use. 2 ml of the thrombin solution was drawn into a new sterile syringe and the syringe was connected to a pre-filled gelatin syringe and the contents were admixed between the two syringes approximately six times.

B. Surgiflo with PEG-SG and PEG-Amine [pH=9.0]

2.5 mL 228 mg/mL PEG-Amine in 50 mM carbonate buffer (pH=9.9) was mixed with one unit of Surgiflo by passing the two components back and forth 10 times. The suspension was transferred to a 20 mL syringe and all air was removed. 2.5 mL 50 mM carbonate buffer (pH=9.0) was added to 750 mg PEG-SG for a concentration of 300 mg/mL PEG-SG. The PEG-SG was dissolved via gentle inversion and allowed to dissolve for five minutes. Once dissolved the PEG-SG solution was transferred to a 20 mL syringe and all air was removed. The PEG-SG solution was mixed with the Surgiflo/PEG-Amine solution by passing the two components back and forth 8 times using the dual syringe method. The sealant was immediately injected into the tensile molds for testing.

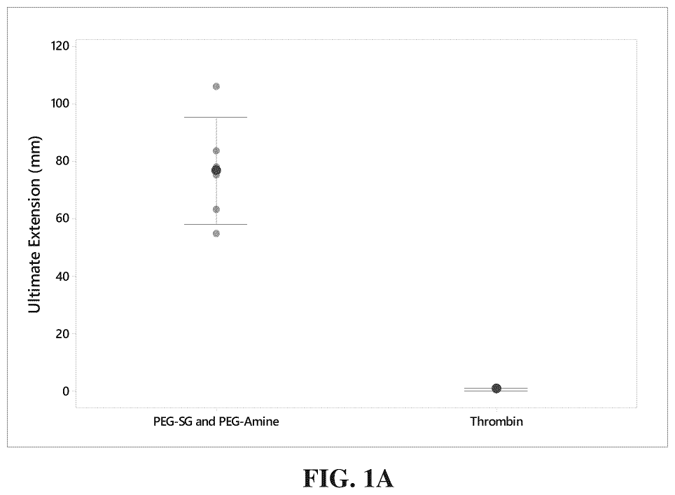

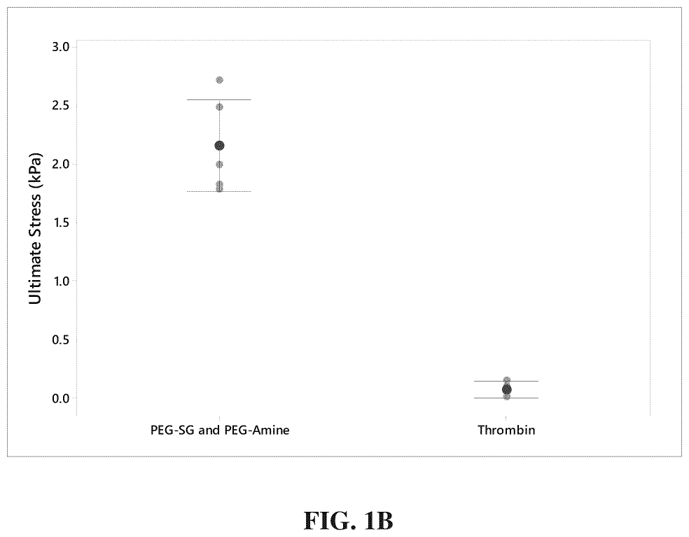

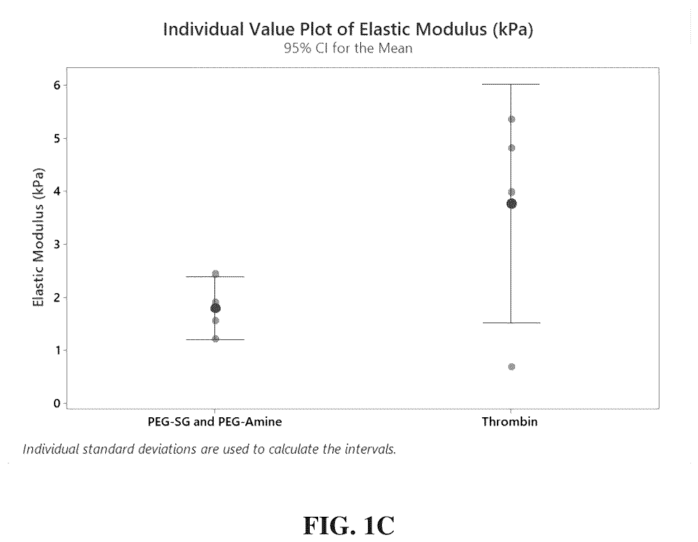

The tensile test measures the elongation at complete failure of the sealant when tested in a vertical, tensile direction at 5 mm/min. The sealant was applied to into custom ‘dog bone’ fixtures and allowed to fully cure. Once cured, the samples were tested in uniaxial tensile testing at 5 mm/min using an Instron Tensile Testing Machine. As shown in FIG. 1A, the ultimate extension of Surgiflo with PEGs was 127-fold significantly greater than Surgiflo with thrombin.

As shown in FIG. 1B, the ultimate stress of Surgiflo with PEGs was 28-fold significantly greater than Surgiflo with thrombin.

As shown in FIG. 1C, the elastic modulus of Surgiflo with PEG, was moderately decreased relative to the Surgiflo with thrombin

In these tests, Surgiflo with thrombin was used as a negative control as there is no expected cohesive strength from this formulation without the presence of blood. As such, Surgiflo with thrombin was used as a baseline to demonstrate that the addition of the PEGs is responsible for the cohesive strength of the sealant formulations.

### Example 3—Viscosity of Gelatin-PEG Composite Flowable Sealants

The purpose of this study was to determine the effect of the addition of gelatin to synthetic poly(ethylene glycol) based sealants. The addition of Surgiflo and Surgifoam gelatin to PEG in situ crosslinking sealants was observed to improve the ability of the sealant to resist extrusion from lung tract defect sites in ex-vivo and in-vivo studies. The ability to resist extrusion can be estimated by analyzing the bubble rise viscosity of leaking air in the lung tract. Terminal bubble rise viscosity (u_{∞}) is described by the following equation:

$${u}_{∞} = \frac{1}{18} \frac{{gd}_{e}^{2} ( {ρ}_{l} - {ρ}_{g} )}{{μ}_{l}}$$

where g is the acceleration due to gravity, dₑ is the equivalent bubble diameter, μl is the dynamic viscosity of the sealant, ρₗ is the density of the sealant, and ρ_{g} is the density of the gas.

Materials:
Sodium Carbonate, Sigma S7795, Lot: BCCB0812
Non-functional PEG, 4 Arm, 20 kDa, Jenkem, Lot: LP2005R-191101

Methods:

In this study, non-functional PEG was utilized as a model compound for PEG-SG and PEG-Amine to avoid gelation during the measurement process. 124 mg/mL non-functional PEG was dissolved in 100 mM carbonate buffer (pH=9.0). 5 mL PEG was mixed with 1 unit of Surgiflo using the dual syringe exchange method with 10 passes. The Surgiflo/PEG mixture was serially diluted using the dual syringe exchange method 1:2 to achieve 50%, 25, and 12.5% units of Surgiflo in PEG. 5 mL PEG was mixed added to 1 unit of Surgifoam powder by vigorously shaking the provided container. The Surgifoam/PEG mixture was serially diluted using the dual syringe exchange method 1:2 to achieve 50%, 25, and 12.5% units of Surgiflo in PEG.

The 100% Surgiflo/PEG and 100% Surgifoam/PEG mixtures were combined in an equal ratio to achieve a 100% Surgifoam/PEG mixture. The Surgiflo/Surgifoam/PEG mixture was serially diluted using the dual syringe exchange method 1:2 to achieve 50%, 25%, and 1.55% units of Surgiflo/Surgifoam in PEG. Note: These concentrations are the sum of Surgiflo and Surgifoam: the concentration of each component is half of the total gelatin concentration.

The composite mixtures were tested using a rheometer in a controlled rate rotational setting. The temperature was held constant at 24° C. 1 mL of sample was added, and the crosshead was lowered at 100/s. After 2 minutes of temperature pre-conditioning, the viscosity was measured at 10 linear steps from 100 to 1000/s.

The densities were calculated based off the values shown below.

TABLE 1

| Sample | Density (g/mL) |
|---|---|
| 124 mg/mL non-functional PEG | 1.124 |
| Surgiflo | 0.7 |
| Surgifoam Powder | 1.52 |

Results:

The density calculations, viscosity data, and resulting relative terminal bubble velocity are shown in the table below.

TABLE 2

| | Concentration | | Dynamic | Relative |
|---|---|---|---|---|
| | (% of | Calculated | Viscosity | Terminal |
| Gelatin | units/ | Density | (Pas) at | Bubble |
| Source | 5 mL) | (g/mL) | 100/s | Velocity |
| Surgiflo | 100% | 0.898 | 1.41 | 0.0058 |
| Surgiflo | 50% | 1.011 | 0.102 | 0.0908 |
| Surgiflo | 25% | 1.068 | 0.0264 | 0.3705 |
| Surgiflo | 12.5% | 1.096 | 0.0168 | 0.5977 |
| Surgiflo | 0% | 1.124 | 0.0103 | 1.0000 |
| Surgifoam | 100% | 1.324 | 1.92 | 0.0063 |
| Surgifoam | 50% | 1.224 | 0.173 | 0.0648 |
| Surgifoam | 25% | 1.174 | 0.108 | 0.0996 |
| Surgifoam | 12.5% | 1.149 | 0.0528 | 0.1994 |
| Surgifoam | 0% | 1.124 | 0.0103 | 1.0000 |
| Surgiflo/Surgifoam | 100% | 1.118 | 1.84 | 0.0056 |
| Surgiflo/Surgifoam | 50% | 1.121 | 0.315 | 0.0326 |
| Surgiflo/Surgifoam | 25% | 1.122 | 0.142 | 0.0724 |
| Surgiflo/Surgifoam | 12.5% | 1.123 | 0.0368 | 0.2797 |
| Surgiflo/Surgifoam | 0% | 1.124 | 0.0103 | 1.0000 |

The terminal bubble velocity data was fit to an exponential curve according to the following equations:

Surgiflo U_{∞}=1.0054e^{-4.2195*[Surgiflo]}

Surgifoam U_{∞}=0.9970e^{-11.9319*[Surgifoam]}

Surgiflo/Surgifoam U_{∞}=0.9999e^{-10.2123}*[Surgiflo/Surgifoam]

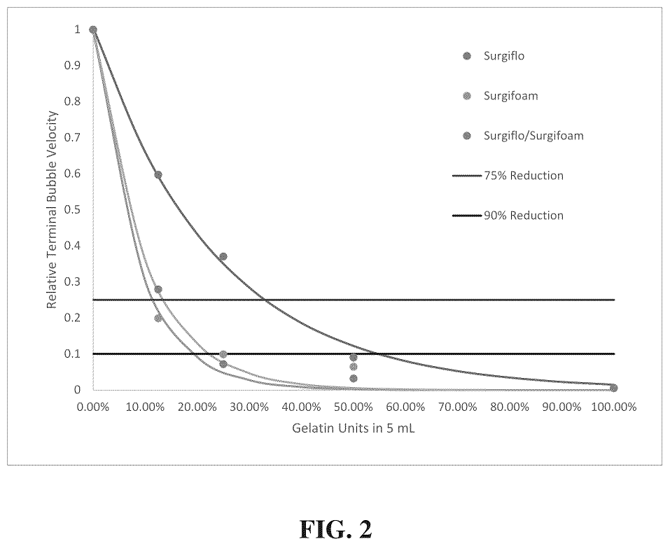

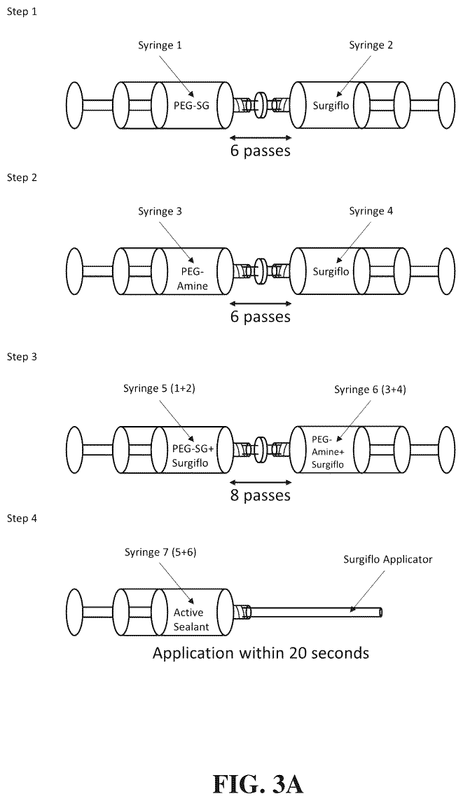

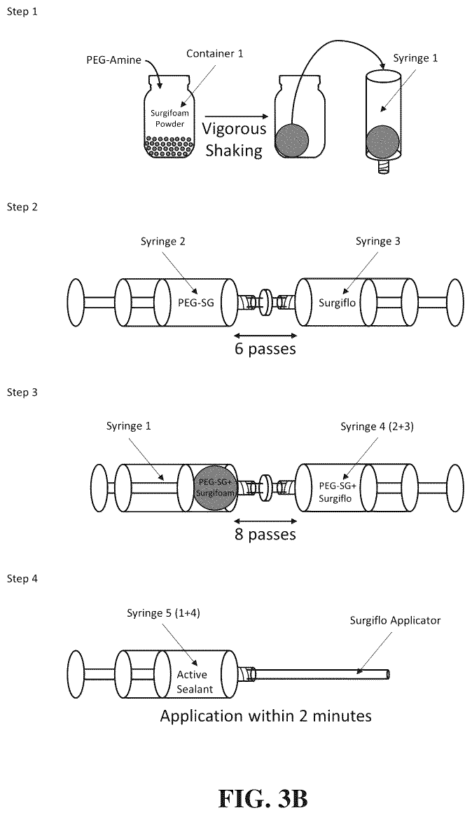

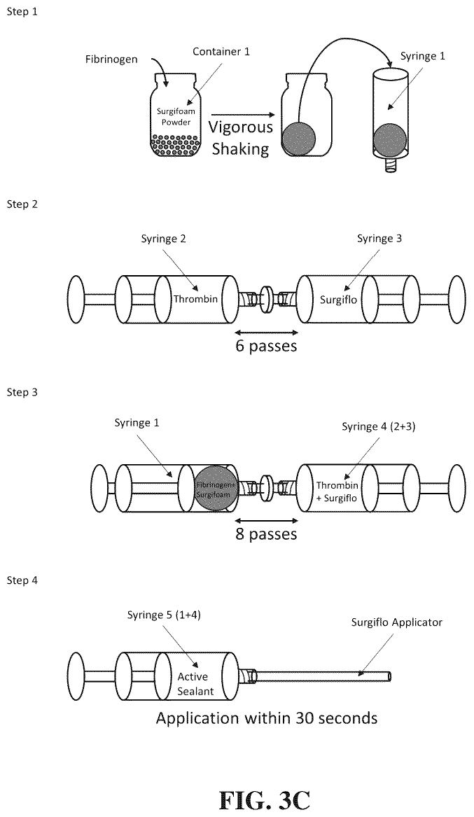

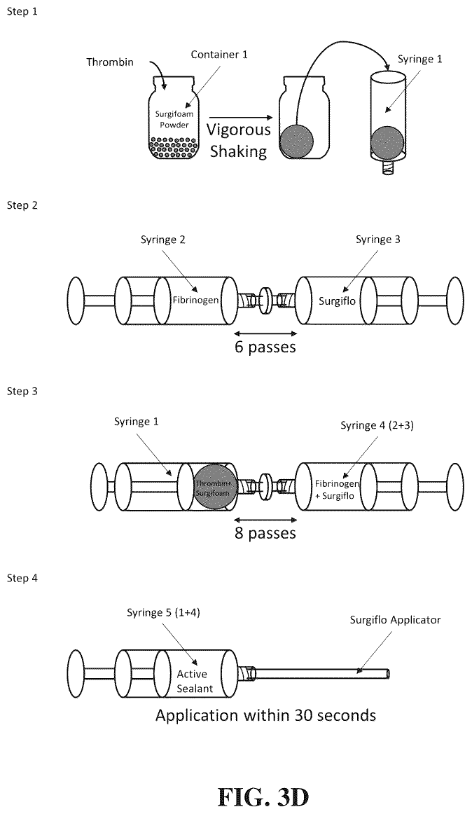

The curve fits shown in FIG. 2 were utilized to determine the concentration at which the terminal bubble rise velocity was decreased by an order of magnitude. The minimum concentrations that correspond to a 90% and 75% reduction can be seen in the table below.

TABLE 3

| | Predicted Concentration ± | Predicted |
|---|---|---|
| | Standard Error | Concentration |
| | (% units/5 mL) | (% units/5 mL) |
| | Required for 90% | Required for 75% |
| | reduction in U_{∞} | reduction in U_{∞} |
| Surgiflo | 54.6 ± 1.0 | 32.9 ± 1.5 |
| Surgifoam | 19.3 ± 3.0 | 11.6 ± 4.5 |
| Surgiflo/Surgifoam | 22.5 ± 0.9 | 13.6 ± 1.4 |

The viscosity of the solution is the primary factor dictating the bubble rise velocity. A minimum concentration of 54.6% units of Surgiflo, 19.3% units of Surgifoam, and 22.5% units of Surgiflo/Surgifoam (or 11.25% of each) per 5 mL sealant improves the ability of the sealant to resist extrusion of air bubbles by 90%. A minimum concentration of 32.9% units of Surgiflo, 11.6% units of Surgifoam, and 13.6% units of Surgiflo/Surgifoam (or 6.8% of each) per 5 mL sealant improves the ability of the sealant to resist extrusion of air bubbles by 75%.

### Example 4—In Situ Setting Gelatin Paste Hydrogel/Hydrocolloid Composite

Materials: 2 units of Surgiflo®, 2.5 mL 300 mg/mL 4-Armed PEG-SG, molecular weight of 20k (pH=9.0), and 2.5 mL 228 mg/mL 4-Armed PEG-Amine, molecular weight of 5k (pH=11.0).

Method of Making

2.5 mL 228 mg/mL 4 Arm PEG-Amine-5k (pH=11.0) was added to 1 unit of Surgiflo® and incorporated using a dual syringe exchange method with at least six passes. The high pH of the PEG-Amine is present to accelerate the crosslinking.

2.5 mL 300 mg/mL 4 Arm PEG-SG-20k (pH=9.0) [Note: The pH of the PEG-SG could not be modified in the same manner as it would result in undesired hydrolysis of the SG group, leading to decreased activity of the crosslinker] was added to 1 unit of Surgiflo® and incorporated using a dual syringe exchange method with at least six passes.

At the point of use, the Surgiflo®-PEG-Amine dispersion and Surgiflo®-PEG-SG dispersion can be combined via the dual syringe exchange method with 8 passes. The sealant has a working time of approximately 20 seconds.

Method of Sealing

The sealant can be applied using the typical Surgiflo® applicator tip and crosslinks within 20 seconds.

The flowability allows for excellent conformance to the tract. The viscosity, density, and rapid crosslinking of the sealant prevents the sealant from being disturbed by the positive pressure of the lung when on ventilation.

Of note with regard to this particular formulation is that this embodiment required high concentrations of PEG-SG and PEG-Amine to reduce the water content of the formulation. When tested at lower concentrations, the formulation had a liquid consistency that was not effective.

### Example 5 In Situ Setting Hydrogel/Hydrocolloid Using Gelatin Paste/Powder Blend

Materials: 1 unit of Surgiflo®, 1 unit of Surgifoam® powder, 5 mL 150 mg/mL 4 Arm PEG-SG-20k (pH=9.0), and 5 mL 114 mg/mL 4 Arm PEG-Amine-5k (pH=9.0).

Method of Making

5 mL 114 mg/mL 4 Arm PEG-Amine-5k (pH=9.0) was added to Surgifoam® powder and incorporated into the powder by vigorously shaking the powder container. Time should be provided to allow PEG-Amine to adsorb onto the gelatin particles via hydrophobic interactions. The PEG-Amine/Surgifoam® Powder mixture creates a ball of material. The plunger is removed from Syringe 1, the ball of material is transferred to Syringe 1, and the plunger is returned.

Syringe 2 is filled with 5 mL 150 mg/mL 4 Arm PEG-SG-20k (pH=9.0). Syringe 3 is filled with 1 unit of Surgiflo®. Syringes 2 and 3 are mixed via the dual syringe exchange method with 6 passes. Syringe 4 is filled with the mixture of PEG-SG and Surgiflo®.

Syringes 1 and 4 are mixed together to activate the sealant via the dual syringe method with 8 passes. The sealant has a 2-minute working time. The sealant has a working time of approximately 2 minutes.

Method of Sealing

The sealant can be applied using the typical Surgiflo® applicator tip and crosslinks within 2 minutes. The Surgifoam® powder provides the ability of the sealant to swell and increases the viscosity. The viscosity and density of the sealant prevents the sealant from being disturbed by the positive pressure of the lung when on ventilation. The longer working time allows for tamponade to be performed as the sealant swells and crosslinks.

### Example 6: In Situ Setting Fibrinin/Gelatin Composite

Materials: one unit of Surgiflo® and one unit of Surgifoam® powder, 5 mL Evicel Fibrinogen, and 5 mL Evicel Thrombin.

Method of Making

5 mL Evicel Fibrinogen was incorporated with 1 unit of Surgifoam® Powder via vigorous shaking in Container 1. Time should be provided to allow fibrinogen to physically interact with the gelatin. The fibrinogen will form a monolayer surrounding the gelatin particles. The Fibrinogen/Surgifoam® Powder mixture created a ball of material. The plunger is removed from Syringe 1, the ball of material is transferred to Syringe 1, and the plunger is returned. Syringe 2 was filled with 5 mL Evicel Thrombin. Syringe 3 was filled with 1 unit of Surgiflo®. Syringes 2 and 3 were mixed via the dual syringe exchange method with 6 passes. Syringe 4 was filled with the mixture of Thrombin and Surgiflo®. Syringes 1 and 4 were mixed together to activate the sealant via the dual syringe method with 8 passes. The sealant has a 30 second working time.

At the point of use, the fibrinogen-soaked powder can be rolled into a ball and transferred to a 20 mL syringe by hand. The Surgiflo®-thrombin dispersion can be then mixed with the fibrinogen-soaked powder via the dual syringe exchange method with 10 passes. The sealant has a working time of approximately 30 seconds (Note: Typically, fibrin sealants have a working time of less than 5 seconds).

Method of Sealing

At the point of use, the fibrinogen-soaked powder can be rolled into a ball and transferred to a 20 mL syringe by hand. The Surgiflo®-thrombin dispersion can be then mixed with the fibrinogen-soaked powder via the dual syringe exchange method with 10 passes. The sealant can be applied using the typical Surgiflo® applicator tip and crosslinks within 30 seconds. The viscosity and density of the sealant prevents the sealant from being disturbed by the positive pressure of the lung when on ventilation.

The resulting plug is a thick paste, can conforms well to the defect when applying a tamponade, and, once crosslinked is tough, elastic, and compressible. The Surgifoam® powder provides a scaffold for the fibrinogen to form, the ability of the sealant to swell, and increases the viscosity.

As a result of the interaction of the fibrinogen with the Surgifoam® powder prior to usage, the gelatin powder is physically interacted with the fibrin network. Another potential benefit of this formulation is when the sealant is exposed to bleeding and endogenous fibrin formation, the sealant will become further stabilized.

### Example 7—In Situ Setting Fibrinin/Gelatin Composite (Low Expression Force Formulation)

Materials: 1 unit of Surgiflo®, 1 unit of Surgifoam® powder, 5 mL Evicel Fibrinogen, and 5 mL Evicel Thrombin.

Method of Making

To make a sealant requiring lower expression force, 5 mL Evicel Thrombin was incorporated with 1 unit of Surgifoam® Powder via vigorous shaking in Container 1. The Thrombin/Surgifoam® Powder mixture creates a ball of material. The plunger was removed from Syringe 1, the ball of material transferred to Syringe 1, and the plunger is returned. Syringe 2 was filled with 5 mL Evicel Fibrinogen. Syringe 3 was filled with 1 unit of Surgiflo®. Syringes 2 and 3 were mixed via the dual syringe exchange method with 6 passes. Syringe 4 was filled with the mixture of Fibrinogen and Surgiflo®. Syringes 1 and 4 were mixed together to activate the sealant via the dual syringe method with 8 passes. The sealant has a 30 second working time.

At the point of use, the thrombin-soaked powder can be rolled into a ball and transferred to a 20 mL syringe by hand. The Surgiflo®-fibrinogen dispersion can be then mixed with the thrombin-soaked powder via the dual syringe exchange method with 10 passes. The sealant has a working time of approximately 30 seconds (Note: Typically, fibrin sealants have a working time of less than 5 seconds).

The resulting plug is a thick paste, can conform well to the defect when applying a tamponade, and, once crosslinked is tough, elastic, and compressible.

Method of Sealing

The sealant can be applied using the typical Surgiflo® applicator tip and crosslinks within 30 seconds. The viscosity and density of the sealant prevents the sealant from being disturbed by the positive pressure of the lung when on ventilation.

This embodiment results in a sealant that requires less expression force to mix using the dual syringe method than the second embodiment. The sealant has a similar flowability, consistency, and working time.

### Example 8—Dehydrated Sealant Formulation

Materials: 1 unit of Surgiflo®, 1 unit of Surgifoam® powder, 5 mL 150 mg/mL 4 Arm PEG-SG-20k, and 5 mL 114 mg/mL 4 Arm PEG-NH2-5k in 100 mM carbonate (pH=9.0).

Method of Making

5 mL 114 mg/mL 4 Arm PEG-Amine-5k (pH=9.0) are added to Surgifoam® powder and is incorporated into the powder by vigorously shaking the powder container. Time should be provided to allow PEG-Amine to adsorb onto the gelatin particles via hydrophobic interactions.

5 mL 150 mg/mL PEG-SG-20k (pH=9.0) are added to Surgiflo® using a dual syringe exchange method with at least six passes.

The Surgifoam®-PEG-Amine dispersion and Surgiflo®-PEG-SG dispersion can be combined via the dual syringe exchange method with 10 passes and expressed into a cylindrical mold with a diameter 0.41 to 1.8 mm.

The formulation is frozen at −80° C. for 1 hour, and then lyophilized, and released from the mold. The resultant plug is stiff and tough and can be utilized to seal lung needle biopsy tracts.

Method of Sealing

Following a lung needle biopsy procedure performed via an introducer needle, a dehydrated plug can be inserted into the tract. The plug is rehydrated from the surrounding tissue fluids and, if necessary, saline. The Surgifoam® powder provides the ability of the plug to swell when rehydrated.

### Example 9: Comparison of Gelatin and Collagen as Swellable Filler

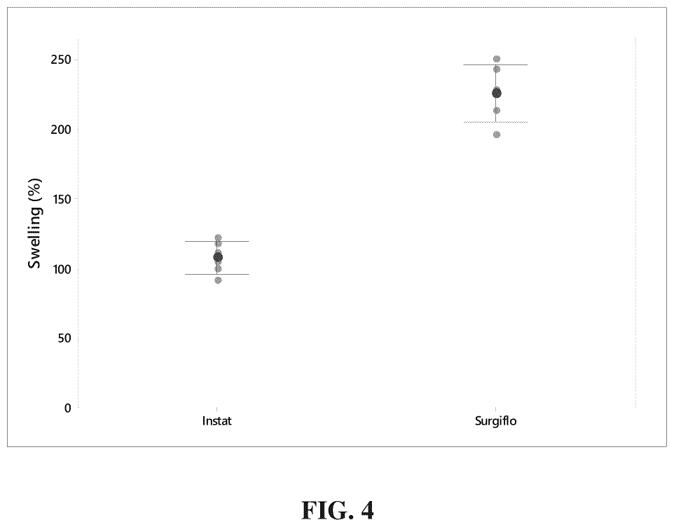

The purpose of this study was to assess the swelling of collagen and gelatin composite hydrogels. In some applications, swelling is an advantageous property that can help seal large leaks. Surgiflo gelatin is known to swell to 35× its original weight in blood and collagen swells to only about 2× its original weight. In this study, collagen and gelatin were compared as the swellable component of a crosslinked PEG based composite hydrogel in regard to swelling. As shown in FIG. 4, the Surgiflo formulation swelled more than twice that of Instat (collagen formulation) on average (225.7 vs 107.7%). There was a significant difference in swelling (p<0.01).

### Example 10: Ex-Vivo Pressure Test

Two sample formulations, one prepared according to Example 5, and one sample prepared according to Example 6, were evaluated for their ability to achieve pneumatosis in an ex vivo porcine large lung tract model.

In this model, lung plucks were harvested fresh on the day of testing and kept moist until testing. Prior to testing, the lungs were placed on a ventilator to recruit collapsed alveoli (goal is to open up collapsed airless alveoli). At the time of testing, lungs were connected to a Respironics respirator to precisely control the pressure during ventilation cycles. The pressure was set to an inspiration pressure of 25 cm water and expiration pressure of 5 cm water (Δ 20 cm water).

Lung defects were created with a coring device with a diameter of 18 mm with resulting puncture size of approximately 20 mm diameter to depth of 3 cm. The air leak in the defect was assessed as severe with a bubble test. When the samples were applied, pressure was reduced to inspiration pressure of 10 cm water and expiration pressure of 10 cm water (no change) to keep the lungs expanded.

After the samples were applied, typically topical compression was placed on the lung surface at the sealant site for 1 min while the lung was still expanded and under positive pressure. To test performance, lung was ventilated starting at low pressure and increasing to inspiration pressure of 25 cm water and expiration pressure of 5 cm water (Δ 20 cm water). Bubble test was performed by passing saline over puncture site and recording for presence and severity of air leak. For an additional challenge, ventilation pressures were increased to inspiration pressure of 40 cm water and expiration pressure of 5 cm water (Δ 35 cm water).

Results

Surgifoam®/Surgiflo®/PEG liquid sealant combination (Example 5). The components were mixed using the dual syringe method to create a uniform paste/plug which was injected into the defect and allowed to polymerize for 3 min with tamponade. No leaks at 35 cm water pressure and the paste adhered well to the surrounding tissue.

Surgifoam®/Surgiflo® Evicel Fibrin Sealant (Example 6). The combination of components was mixed into a paste and injected directly into the defect created using a coring device. Topical compression was applied to the paste for 3 min while the lung was expanded and under positive pressure. No leaks were observed at 20 cm water. The sealant adhered well to the surrounding tissue. This same formulation was also tested on a different lung during the same testing occasion and was also successful in sealing a defect created with a 20 mm biopsy punch.

### Example 11: Ex Vivo Pneumostasis Testing

Needle tract sealing prototypes were assessed in an ex vivo porcine lung model. The goal of the testing was to evaluate pneumostasis effectiveness of pre-formed plug/paste sealant prototypes to close a pleural and parenchymal lesion in the lung after a percutaneous or thoracoscopic needle lung biopsy. Lung plucks were freshly harvested on the day of testing. Immediately, prior to testing, the lungs were placed on a ventilator to recruit collapsed alveoli. The lungs were connected to a Respironics respirator to precisely control the pressure during ventilation cycles. The pressure was set to an inspiration pressure of 25 cm water and expiration pressure of 5 cm water (Δ 20 cm water) to acclimate lungs.

During needle biopsies, the lungs were expanded by setting the respirator to a constant pressure of 10 cm water (inspiration and expiration pressure of 10 cm water). The needle tracts were created in the lungs using a 19-gauge biopsy needle that was inserted through a coaxial needle port which was positioned 3 cm deep. Prototype plugs were either inserted into the needle tract using the Biosentry plug assembly and stylet or were inserted manually by pushing the plug into position using the stylet.

After prototype application/insertion, a time duration of at least 3 min was allowed for the prototype to expand and/or polymerize within the lung while under positive pressure (10 cm water). To test sealing performance, lung was ventilated at 20 cm water pressure differential (25 cm water inspiration pressure and 5 cm water expiration pressure, i.e., Δ 20 cm water). A bubble test with saline was performed to assess the presence and severity of any air leak. The results obtained for specific prototypes are shown below.

Results
Prototype 1: Lyophilized Surgifoam®/Surgiflo® PEG Liquid Plug

Minor leak observed at 20 cm water pressure. Leak was significantly reduced relative to untreated needle tract defect.
Prototype 2: Lyophilized Evicel Fibrin Sealant Plug

Minor leak observed at 20 cm water pressure. Leak was significantly reduced relative to untreated needle tract defect.
Prototype 3: Lyophilized Biosynthetic liquid (PEG-SG4+Albumin) Plug

No leaks observed at 20 cm water pressure.
Prototype 4: Lyophilized Biosynthetic Foam Plug (2:1 Liquid to air)

Minor leak observed at peak pressure when ventilated at 20 cm water pressure. Leak was significantly reduced relative to untreated needle tract defect.
Prototype 5: Surgiflo®/Evicel Fibrin Sealant paste delivered with 18-gauge needle

The paste was prepared with 10 mL of Evicel and 2 units Surgiflo®. After paste application, tamponade was held for 3 min to allow clotting. This formulation was tested at two needle track defects sites, and both successfully achieved pneumostasis at 20 cm and 35 cm water pressure.
Prototype 6: Surgiflo®+thrombin (standard paste formulation without fibrinogen) delivered with 18-gauge needle.

After paste application, tamponade was held for 3 min. This formulation successfully achieves pneumostasis at 20 cm water pressure, however, was not able to stop the air leak at 35 cm water pressure.

### Example 12: In Vivo Test

The Surgiflo® Sealant with Reactive PEG in situ prototype was tested in a live porcine animal model.

2.5 mL 228 mg/mL PEG-Amine-5k, 50 mM carbonate (pH=11.0) was combined with one unit of Surgiflo® via the dual syringe exchange method with 10 passes. The mixture was transferred to a 20 mL syringe. 2.5 mL 300 mg/mL PEG-SG-20k, 50 mM carbonate (pH=9.0) was combined with one unit of Surgiflo® via the dual syringe exchange method with 10 passes. The mixture was transferred to a 20 mL syringe. At the time of application, the two syringes containing the Surgiflo®/PEG mixtures were connected with a dual syringe connector and passed 8 times. The syringe was connected to a Surgiflo® tip and completely expressed within 10 seconds.

A coring device was used to core a defect. Once the defect was created, the chest cavity was opened. The chest wall distance was measured as 5 cm in length. The defect length in the lung tissue was 4.5 cm. Nearly no bleeding was observed. The Surgiflo®/PEG sealant was prepared and the PEG-SG was hydrated for seven minutes before use. The lung was maintained at approximately 10 cm H₂0 constant pressure. The entire volume of the Surgiflo®/PEG sealant was expressed into the defect. The sealant flowed into the defect easily and conformed very well to the defect site.

After 1 minute, no air leak was observed via the bubble test at full ventilation. The sealant achieved homeostasis and pneumostasis.

What is claimed:

1. A hydrogel-hydrocolloid expandable soft tissue sealant composition comprising:
a cross-linkable electrophilic component;
a cross-linkable nucleophilic component;
a swellable filler material including gelatin; and,
a buffer having a pH in the range of about 9.0 to about 10.0;
wherein the composition has a first fluid state and a second crosslinked state, and wherein in the crosslinked state, the electrophilic and nucleophilic components crosslink to form a crosslinked hydrogel network; and,
wherein the swellable filler material including gelatin is disposed within the crosslinked network.

2. The composition of claim 1, wherein the gelatin is thermally crosslinked.

3. The composition of any one of claim 1 or claim 2, wherein the gelatin is a prewet gelatin fluid.

4. The composition of any one of claim 1 or claim 2, wherein the gelatin is dry gelatin particles.

5. The composition of any one of claim 1 or claim 2, wherein the swellable filler material includes a blend of prewet gelatin fluid and dry gelatin particles.

6. The composition of any one of the preceding claims, wherein the swellable filer material is present in the in the composition in an amount in the range of 50 mg/ml to about 10 mg/ml.

7. The composition of any one of the preceding claims, wherein in the crosslinked state the composition has a terminal bubble velocity, and wherein the terminal bubble velocity of the crosslinked composition is at least 75% less than a terminal bubble velocity of a composition including the electrophilic and nucleophilic components in the absence of the swellable filler.

8. The composition of claim 7, wherein the terminal bubble velocity of crosslinked the composition is at least 90% less than a terminal bubble velocity of a composition including the electrophilic and nucleophilic components in the absence of the swellable filler.

9. The composition of any one of the preceding claims, wherein at least one of the cross-linkable electrophilic component and the cross-linkable nucleophilic component is a synthetic compound.

10. The composition of claim 9, wherein both the cross-linkable electrophilic component and the cross-linkable nucleophilic component are synthetic compounds.

11. The composition of claim 10, wherein the electrophilic component includes a multi-arm polyethylene glycol (PEG) based polymer, and wherein the nucleophilic component includes a multi-arm PEG polymer containing at least one reactive amine group.

12. The composition of any one of claims 9-11, wherein the electrophilic component includes a PEG N-hydroxysuccinimide activated ester (PEG-NHS).

13. The composition of claim 12, wherein the PEG-NHS is a PEG-succinimidyl glutarate ester (PEG-SG).

14. The composition of any of claims 1-8, wherein at least one of the cross-linkable electrophilic component and the cross-linkable nucleophilic component is a biological compound.

15. The composition of claim 14, wherein both the cross-linkable electrophilic component and the cross-linkable nucleophilic component are biological compounds.

16. The composition of claim 15, wherein the electrophilic compound is thrombin and the nucleophilic compound is fibrinogen.

17. The composition of any one of the preceding claims, wherein the composition is in the crosslinked state and a solid, and further wherein the composition is lyophilized.

18. The composition of any one of the previous claims, wherein the swellable filler material is configured to expand in volume in the crosslinked state such that the composition has a crosslinked expandable state, and wherein in the crosslinked expandable state, the composition is configured to provide a fluid tight seal at a pressure differential of up to 35 cm of water.

19. A system for forming a hydrogel-hydrocolloid expandable soft tissue sealant comprising:
a first container containing a cross-linkable electrophilic component; and,
a second container containing a cross-linkable nucleophilic component; and,
a swellable filler material including gelatin, wherein the swellable filler material is disposed in either the first container, the second container, or both;
wherein the first container and the second container are configured to connect in fluid communication with one another; and,
wherein the cross-linkable electrophilic component and the cross-linkable nucleophilic component are configured to form a cross-linked hydrogel network upon admixture between the first container and the second container, and the swellable material is disposed within the crosslinked hydrogel network.

20. The system of claim 19, wherein the swellable material is disposed in the first container.

21. The system of claim 19, wherein the swellable material is disposed in the second container.

22. The system of claim 19, wherein the swellable material is disposed in each of the first and second container.

23. The system of claim 19, wherein the swellable material is a prewet gelatin fluid.

24. The system of claim 19, wherein the swellable material is dry gelatin particles.

25. The system of claim 19, wherein the swellable material includes both prewet gelatin fluid and dry gelatin particles.

26. The system of claim 19, wherein both the first and second containers contain prewet gelatin fluid.

27. The system of claim 19, wherein the first container contains prewet gelatin fluid and the second container contains dry gelatin particles.

28. The system of claim 19, wherein the first container contains dry gelatin particles, and the second container contains prewet gelatin fluid.

29. A method of repairing a soft tissue defect comprising:
applying a hydrogel-hydrocolloid expandable sealant composition to a soft tissue defect, the composition containing a first cross-linking component and a second crosslinking component, and a swellable filler material including gelatin;
wherein the soft tissue defect extends from an outer surface of the soft tissue to an inner surface of the soft tissue, and wherein the inner surface defines a void containing a fluid exerting a positive pressure in the direction from the void to the outer surface;
exposing the sealant composition to an aqueous fluid such that the composition will crosslink and swell such that the composition will fluidly seal the soft tissue defect with adhesion to the soft tissue surface and expansive forces across the defect.
